(19) United States
(12) Patent Application Publication
(10) Pub. No.: US 2018/0250368 A1
(43) Pub. Date: Sep. 6, 2018

(54) LIQUID COMPOSITION OF HUMAN ALBUMIN FOR THERAPEUTIC USE

(71) Applicant: LABORATOIRE FRANCAIS DU FRACTIONNEMENT ET DES BIOTECHNOLOGIES, Les Ulis (FR)

(72) Inventors: Damien BATAILLE, Ormoy (FR); Michel NOGRE, Vanves (FR); Guillaume CHEVREUX, Paris (FR)

(21) Appl. No.: 15/759,357

(22) PCT Filed: Sep. 9, 2016
(86) PCT No.: PCT/FR2016/052274

§ 371 Date: Mar. 12, 2018

(30) Foreign Application Priority Data
Sep. 10, 2015 (FR) 1558422

(51) Int. Cl. A61K 38/38; A61K 9/19

(52) U.S. Cl. CPC A61K 38/385; A61K 9/19

(57) ABSTRACT

Disclosed is a liquid composition of human albumin for therapeutic use, in which at least 50% of the albumin has a molecular weight of 66.438 Da, plus or minus 5 Da, preferentially plus or minus 2 Da, the composition also including a sodium salt.

The invention relates to an enhanced-activity liquid composition of human albumin for therapeutic use.

### BACKGROUND OF THE INVENTION

Albumin is a highly-soluble plasma protein produced by the liver. Its primary structure consists of a polypeptide chain of 585 amino acids, including 35 cysteine residues, only one of which (cysteine 34, or Cys-34) is in the reduced state. Daily albumin synthesis amounts to about 120 mg·kg⁻¹, thus renewing about 5% of the protein each day. Albumin represents more than 50% of the blood plasma proteins in humans and animals. This plasma protein is known to have a multitude of beneficial roles. In particular, albumin helps maintain oncotic pressure by maintaining the proper distribution of liquids between the blood vessels and the tissues or the interstitial fluid. Albumin also plays a part in the transport of various endogenous substances, such as fatty acids, metal ions (copper, zinc), thyroid and steroid hormones, and amino acids (chiefly tryptophan and cysteine); and of exogenous substances, such as vitamins and medicinal substances (ibuprofen, diazepane, etc.). This binding capacity also allows albumin to play the role of neutralizer of toxic substances (benzene, aflatoxin GI, etc.), which it sequesters and renders harmless to the organism. Moreover, albumin has antioxidant properties mainly owing to its capacity to bind numerous ligands and to the free thiol group at Cys-34, which further enables it to trap free radicals.

Albumin is already used in the treatment of several diseases for its oncotic and colloidal properties. For example, albumin is used as emergency treatment to restore or maintain circulating blood volume in patients in hypovolemic shock. It is also used in the context of the treatment of cirrhosis, extensive burns, adult respiratory distress, haemolytic disease of the newborn, etc.

Furthermore, its antioxidant and anti-free radical properties make it a potentially useful pharmacological molecule for treating, a great many other diseases. For example, recent studies have shown the usefulness of administering albumin in the context of treating various diseases, such as chronic ischaemic heart failure (Ellidag el al. Redox Report 2014; Vol, 0. N.O: 1-6), ischaemic and cardioembolic strokes (Xu W-H et al. Stroke 2014; 45: 00-00), familial amyloidotic polyneuropathy (Kugimiya T et al. 2011; Laboratory Investigation 1-10), systemic lupus erythematosus (Sheikh Z et al. Autoimmunity 2007; 40(7): 512-520), diabetes (Koga M et al. The Journal of Medical Investigation 2013; 60; 40-45), chronic kidney disease (Matsumaya, Y. Clin Expl. Nephrol. 2009), hypertension (Oda E. Intern Med 2014; 53: 655-660), liver diseases in general (Arroyo V. et al. Journal of Hepatology 2014.04.012), and Alzheimer's disease (Cankurtaran et al. JAD, 2012—Stanyon et al. J Biological Chemistry. 2012—Milojevic J et al. Journal of Alzheimer's Disease 38 (2014) 753-765).

It has been shown that albumin present in the blood plasma, although mainly in native undegraded form, can also be in oxidized and/or truncated form. In the unoxidized (or reduced) form, Cys-34 has a free SH group. Conversely, the oxidized form of albumin generally has a sulphinic (R—SO₂H) or sulphonic (R—SO3H) group at Cys-34. The truncated forms of albumin, for their part, have a C-terminal and/or N-terminal end truncated of one or more amino acids. Certain albumins can be in oxidized and truncated form. These in vivo modifications of the albumin molecule are explained primarily by post-translational modifications due to oxidative stress. However, conversion of the reduced form to certain oxidized forms is irreversible, causing the molecule to irrevocably lose its antioxidant properties. Moreover, the truncated forms also have a reduced activity compared with the native form. For example, the loss of N-terminal aspartate and alanine residues deprive the albumin molecule of its capacity to bind copper and free radicals.

However, the albumin compositions for therapeutic use available to date prove to contain only a small proportion of unoxidized and untruncated albumin. The efficacy of these compositions is thus not optimal (Vincent et al., Critical Care 2014, 18(4):231 “Albumin administration in the acutely ill: what is new and where next?”).

There is therefore a need for enhanced compositions based on therapeutically-active human albumin.

### SUMMARY OF THE INVENTION

Surprisingly, it appeared to the Applicant that it is possible to preserve the albumin purified from a human albumin-rich solution by eliminating from the purification process all or part of the ethanol precipitation steps. The Applicant was thus able to purify albumin from blood plasma while retaining its native, unoxidized and untruncated form, and to prepare human albumin-based compositions for therapeutic use mainly comprising such a native albumin. The albumin compositions according to the invention thus have an enhanced antioxidant and transport activity compared with the currently available compositions.

The object of the invention is thus a human albumin composition for therapeutic use wherein at least 50 wt % of the albumin is in native form, i.e., has a molecular weight of about 66,438 Da, i.e., 66,438 Da±5 Da, preferentially+2 Da, said composition further comprising at least one sodium salt.

The composition according to the invention thus comprises at least 50 wt % albumin having a molecular weight of 66,433 Da to 66,443 Da, preferentially of 66,436 Da to 66,440 Da.

The albumin used to prepare such a composition is advantageously obtained by a purification process with no ethanol precipitation step. In another example, the albumin is obtained by a purification process comprising only one ethanol precipitation step, said step being carded out with a low concentration of ethanol, i.e., lower than 10% ethanol. This step advantageously makes it possible to extract fibrinogen from the composition.

Another object of the invention is such a composition for use in the treatment of renal impairment, hepatic impairment, neurodegenerative disease, hypovolemic shock, adult respiratory distress syndrome (ARDS), haemolytic disease of the newborn.

### BRIEF DESCRIPTION OF THE FIGURES

FIG. 1: Deconvolution of the mass spectrum of albumin on the eluate, after the positive chromatography step (HEA HyperCel);

FIG. 2: Deconvolution of the mass spectrum of albumin after the ion-exchange chromatography step (Macro-Prep DEAE NA+L);

FIG. 3: Deconvolution of the mass spectrum of albumin after the mixed-mode chromatography step (MEP HyperCel NA);

FIG. 4: Deconvolution of the mass spectrum of albumin after the heat treatment step;

FIG. 5: SDS-PAGE gel of the successive products from the plasma fraction to the heat-treated product comprising more than 90% albumin, obtained by the multicolumn continuous extraction process.

FIG. 6: Mass spectrum of an albumin composition of the state of the art (Albuminar® from CSL);

FIG. 7: Mass spectrum of an albumin composition according to the invention.

### DETAILED DESCRIPTION

Definitions

In the context of the invention, “native albumin” means human albumin in unoxidized and untruncated form, i.e., having 585 amino acids among which the cysteine residue at position 34 from the N-terminal end comprises a reduced free SH group.

The term “oxidized albumin” is used to indicate an albumin having no SH group at Cys-34. Oxidized albumin notably includes cysteinylated albumin (the Cys-34 of which is attached to a cysteine).

A “truncated albumin”, in turn, refers to an albumin whose N-terminal and/or C-terminal end is truncated, i.e., it lacks at least one amino acid at one end of the protein.

It is possible to easily discriminate the various forms of albumin by the molecular weight. Indeed, native albumin has a molecular weight of about 66,438 Da. Conversely, a truncated albumin has a molecular weight below 66,400 Da, while an oxidized albumin has a molecular weight generally above 66,450 Da. The measurement methods used to determine the molecular weight of the albumin molecule have sufficient precision to discriminate variations due to oxidations or truncations. Preferably, the molecular weight of the albumin molecule is measured by mass spectrometry, preferentially by electrospray mass spectrometry. For example, mass spectrometry analysis is carried out on an ESI-Q-TOF apparatus (Synapt, Waters). If necessary, the protein of interest is first subjected to positive chromatography then eluted, analysis by mass spectrometry then being performed on the eluate (Marie A L et al. “Capillary zone electrophoresis and capillary electrophoresis-mass spectrometry for analyzing qualitative and quantitative variations in therapeutic albumin”, Anal Chim Acta. Oct. 2013 24; 800:103-10. doi: 10.1016/j.aca.2013.09.023. Epub 2013 Sep. 14.).

An “albumin-rich” composition refers to a composition of biological origin, such as blood plasma or transgenic milk, originally comprising a high concentration of albumin, and notably at least 30%, preferentially at least 40%. An “albumin-enriched” composition refers to a composition whose albumin concentration has been increased following a treatment step, in particular a purification step, such as chromatography.

Generally, and unless otherwise specified, percentages refer to weight percentages base on the total weight of the given element.

In the context of the invention, the expression “about” includes the given value ±5, and preferentially ±2.

While working on a new process for fractionation of plasma proteins, the Applicant showed that removing certain ethanol fractionation steps during the purification of albumin from blood plasma, or from any other human albumin-rich solution, makes it possible to preserve the native form of the molecule. Thus, it is possible to obtain an albumin concentrate mainly comprising albumin in native form, by subjecting the albumin-rich solution exclusively to successive chromatography steps. The chromatographies can indifferently be ion-exchange chromatographies or affinity chromatographies. It is also possible to alternate ion-exchange chromatographies and affinity chromatographies. The albumin concentrate thus obtained can then be used to prepare the human albumin-based pharmaceutical composition according to the invention.

Advantageously, the albumin composition according to the invention is obtained by a purification process comprising no precipitation step.

In a particular embodiment of the invention, the albumin composition according to the invention is obtained by a purification process comprising one or more ethanol precipitation steps carried out with a low concentration of ethanol, advantageously lower than 50% ethanol, lower than 45% ethanol, lower than 40% ethanol, lower than 35% ethanol, lower than 30% ethanol, lower than 25% ethanol, lower than 20% ethanol, lower than 15% ethanol, lower than 10% ethanol, lower than 5% ethanol.

In an advantageous embodiment of the invention, the albumin composition according to the invention is obtained by a purification process comprising one or more ethanol precipitation steps carried out with a low concentration of ethanol, advantageously lower than 45% ethanol.

In an advantageous embodiment of the invention, the albumin composition according to the invention is obtained by a purification process comprising one or more ethanol precipitation steps carried out with a low concentration of ethanol, advantageously lower than 30% ethanol.

In an advantageous embodiment of the invention, the albumin composition according to the invention is obtained by a purification process comprising one or more ethanol precipitation steps carried out with a low concentration of ethanol, advantageously lower than 10% ethanol.

In a particular embodiment, the albumin used in the pharmaceutical composition according to the invention has been obtained by a process of purification of a human albumin-rich solution comprising successively a positive chromatography step and a negative chromatography step. The positive chromatography makes it possible, first, to retain the albumin on the column, to remove most of the impurities, then the second chromatography makes it possible, second, to retain on the column the remaining fraction of impurities and to collect the albumin-enriched fraction.

Advantageously, in order to further reduce the risks of denaturing the protein, any cryoprecipitation step can be also eliminated from the albumin purification process. Thus, if the albumin is purified from blood plasma, the chromatographies are advantageously performed directly from said plasma rather than from cryosupernatant.

In a particular embodiment, the human albumin composition for therapeutic use according to the invention has been obtained by a blood plasma fractionation process comprising the steps consisting in:
a) Subjecting the blood plasma to anion-exchange chromatography at pH 5 or higher, advantageously at pH 6 or higher, 7 or higher, 8 or higher;
b) Subjecting the eluted fraction derived from step a) to a first ion-exchange or mixed-mode chromatography,
c) Subjecting the unretained fraction derived from step b) to a second ion-exchange or mixed-mode chromatography, and optionally
d) Subjecting the unretained fraction derived from step c) to heat treatment, in particular to pasteurization, at a temperature of 50° C. to 70° C., preferentially of about 60° C., for a period of 1 hour to 15 hours, preferentially of at least 10 hours.

In a particular embodiment, the human albumin composition for therapeutic use according to the invention has been obtained by a blood plasma fractionation process comprising the steps consisting in:
a) Subjecting the blood plasma to anion-exchange chromatography at pH 6,
b) Subjecting the eluted fraction derived from step a) to a first ion-exchange or mixed-mode chromatography,
c) Subjecting the unretained fraction derived from step b) to a second ion-exchange or mixed-mode chromatography, and optionally
d) Subjecting the unretained fraction derived from step c) to pasteurization, at a temperature of 50° C. to 70° C., preferentially of about 60° C., for a period of 1 hour to 15 hours, preferentially of at least 10 hours.

In a particular embodiment, an ultrafiltration step is carried out before chromatography step a).

In a particular embodiment, a formulation step such as pasteurization is carried out before heat treatment step d).

Advantageously, the elution of the column before step b) is performed at a pH above 3.5, and notably at a pH of about 4.

In another particular embodiment, the human albumin composition for therapeutic use according to the invention has been obtained by a blood plasma cryosupernatant fractionation process comprising the steps consisting in:
a) Subjecting the cryosupernatant to anion-exchange chromatography at pH 5 or higher, advantageously at pH 6 or higher, 7 or higher, 8 or higher;
b) Subjecting the eluted fraction derived from step a) to a first ion-exchange or mixed-mode chromatography,
c) Subjecting the unretained fraction derived from step b) to a second ion-exchange or mixed-mode chromatography, and optionally
d) Subjecting the unretained fraction derived from step c) to heat treatment, in particular to pasteurization, at a temperature of 50° C. to 70° C., preferentially of about 60° C., for a period of 1 hour to 15 hours, preferentially of at least 10 hours.

According to the invention, it is also possible to purify the albumin from a recombinant human albumin concentrate. In a particular embodiment, the albumin is purified from transgenic milk of a non-human mammal. Advantageously, the purification process comprises an initial delipidation step before any chromatography step. In another embodiment, the albumin is purified from a culture supernatant of recombinant cells expressing human albumin.

Generally, the albumin concentrate derived from these purification processes is subjected to one or more viral inactivation steps, such as pasteurization and/or nanofiltration.

In an advantageous embodiment of the invention, the albumin concentrate is subjected to a pasteurization step. In a particular embodiment of the invention, the pasteurization step is performed under conditions allowing to limit N-terminal and C-terminal cleavage and/or to limit cysteinylation phenomena. Thus, the pasteurization step is advantageously performed while maintaining a constant temperature (about 60° C.) throughout the pasteurization step (about 10 h) and/or by controlling the addition of stabilizing excipients such as caprylate and/or tryptophanate.

At the end of the fractionation process, the albumin composition is advantageously subjected to a step of distribution into storage and/or administration containers, such as bottles, bags, syringes, or any other device for storing the product and/or for administering the product to patients.

In an advantageous embodiment of the invention, the distribution step is carried out so as to limit phenomena of oxidation of the albumin composition. For example, the distribution step is carried out in controlled atmosphere, advantageously in the absence of oxygen and/or in the presence of an inert gas such as nitrogen. In a particular embodiment of the invention, the distribution into storage and/or administration containers comprises a step of inerting (replacing the air contained in the dead volume of the container with an inert gas, preferentially nitrogen) and/or of capping under inert gas, for example under nitrogen.

In an advantageous embodiment of the invention, the container for storing and/or administering the albumin composition advantageously consists of oxygen-impermeable materials.

The albumin derived from such purification processes is mostly collected in native form, i.e., unoxidized and untruncated form. It is thus possible to prepare albumin compositions for therapeutic use having a very high antioxidant and anti-free radical activity compared with the currently available albumin compositions.

Thus, the invention proposes a liquid composition of human albumin for therapeutic use comprising human albumin and a sodium salt, at least 50 wt % of said albumin being in native form, i.e., having a molecular weight of about 66,438 Da. In a particular embodiment, at least 60 wt %, preferentially at least 65 wt % of said albumin is in native form, and preferentially at least 70 wt %, 75 wt %, 80 wt %, 85 wt %, 90 wt %, 95 wt %, 99 wt % of said albumin is in native form.

Depending on need, the composition can comprise between 10 and 300 mg/mL albumin, or between 1 and 30 wt % albumin based on the total weight of the composition. Notably, the composition can comprise in a conventional manner between 40 and 50 mg/mL or between 200 and 250 mg/mL albumin. However, insofar as most of the albumin present in said composition has an enhanced activity compared with the albumin of the currently available compositions, lower concentrations may be advantageous for the treatment of certain pathologies, notably of the order of 10, 15, 20, 25, 30 or 35 mg/mL.

According to the invention, the composition advantageously comprises less than 30% albumin in oxidized form and/or less than 20% truncated albumin.

In a particular embodiment of the invention, the composition advantageously comprises less than 30% albumin in oxidized form, preferentially less than 25% albumin in oxidized form and preferentially less than 22%, 20%, 18%, 15%, 10%, 5% albumin in oxidized form.

In another particular embodiment of the invention, the composition advantageously comprises less than 20% truncated albumin, preferentially less than 15% albumin in oxidized form and preferentially less than 12%, 10%, 8%, 5% albumin in truncated form.

In an advantageous embodiment of the invention, the albumin composition further comprises pharmaceutically acceptable excipients, in particular excipients for limiting oxidation phenomena, such as the body's natural antioxidants, in particular N-acetyl-methionine, N-acetyl-methionine sodium octanoate, reduced glutathione, uric acid, alpha-lipoic acid, disodium EDTA, flavonoids, in particular flavonols such as rutin, which quench, or neutralize, reactive oxygen species, such as type I and II free radicals.

The albumin composition of the invention further comprises at least one sodium salt, preferentially selected from sodium chloride, sodium caprylate, sodium carbonate and sodium acetyltryptophanate.

In a particular embodiment, the human albumin composition for therapeutic use according to the invention comprises between 2.5 and 3.5 mg/mL sodium salt.

A particular object of the invention is a human albumin composition for therapeutic use comprising water, about 40 mg/mL albumin and about 3.5 mg/mL sodium salt, preferentially sodium chloride and sodium caprylate, wherein at least 60% of the albumin has a molecular weight of about 66,438 Da.

Another object of the invention is a human albumin composition for therapeutic use comprising water, about 50 mg/mL albumin and about 3.5 mg/mL sodium salt, preferentially sodium chloride and sodium caprylate, wherein at least 60% of the albumin has a molecular weight of about 66,438 Da.

Another object of the invention is a human albumin composition for therapeutic use comprising water, about 200 mg/mL albumin and about 3.5 mg/mL sodium salt, preferentially sodium chloride and sodium caprylate, wherein at least 60% of the albumin has a molecular weight of about 66,438 Da.

If the albumin of the composition according to the invention is derived from blood plasma, the composition can further comprise one or more plasma proteins among transferrin, immunoglobulin G and haptoglobin, generally co-purified with the albumin. Transferrin and haptoglobin can advantageously enhance the antioxidant nature of the albumin composition without inducing notable side effects, whereas immunoglobulin G can limit bacterial or viral infections.

The albumin composition according to the invention is preferentially an aqueous solution.

The albumin composition of the invention is useful in therapy, and notably in injectable form, via the intravenous route.

The invention also relates to a human albumin composition for therapeutic use as described above for use in the treatment and/or the prevention of numerous diseases, in particular renal impairments, hepatic impairments, neurodegenerative diseases, hypovolemic shocks, adult respiratory distress syndromes (ARDS), haemolytic diseases of the newborn.

Owing to its enhanced antioxidant and anti-free radical activity, the composition according to the invention is particularly useful for the treatment or the prevention of neurodegenerative diseases, in particular Alzheimer's.

Similarly, because the N-terminal end of the albumin is retained, the composition according to the invention can be particularly effective in the treatment and the prevention of diseases linked to heavy metals, such as Menkes disease in which affected patients lack copper transporter, or Wilson disease in which affected patients have excess copper in the tissues.

The following examples illustrate the invention without limiting its scope.

### EXAMPLES

### Example 1

### Preparation of an Albumin Concentrate from Blood Plasma

Principle

In this example, plasma albumin is adsorbed after immunoglobulin depletion on a chromatography support whose chemical ligand is a primary amine: HEA-HyperCel gel.

IgG-depleted plasma, collected at the end of the IgG purification process according to a continuous extraction process by multicolumn affinity chromatography, is used, the albumin not binding at all to the affinity gel used.

The IgG-depleted plasma was obtained according to the following steps:

Bags of human plasma used to constitute a roughly 30-L plasma pool comprising between 8 and 10 g/L IgG were thawed in a 37° C. water bath without the product exceeding the internal temperature of 25° C. in order to extract the immunoglobulins by multicolumn affinity chromatography.

The composition of the buffer solutions used during the various steps of the multicolumn affinity chromatography process is summarized in Table 1 below.

TABLE 1
Multicolumn affinity chromatography buffer solutions

| Phases | Composition | Target values |
|---|---|---|
| Equilibration and Wash | 10.0 mM sodium citrate, | pH 7.4 |
| | 100 mM NaCl | |
| Pre-elution | 10 mM sodium citrate, | pH 7.4 |
| | 2.0M NaCl | |
| Elution | Acetic acid | sq pH 3.0 |
| Eluate adjustment | 1M sodium hydroxide | sq pH 4.8 |

For the multicolumn chromatography, a CaptureSelect FcXL affinity gel from Life Technologies (product 19432801L, batch 200814-03) was used. The affinity ligand used is a ligand from BAC (Bio Affinity Company), which specifically binds to the CH₃ domain of the four human IgG subclasses.

Four radial columns from Proxcys (MD 122 MK III—gel volume: 250 mL per column for a total affinity gel volume of 1.0 L of gel; gel height: 12 cm; ratio of inlet diameter to outlet diameter: 2:1) were used in combination with a BioSc Pilot M automated system (Novasep).

The four columns were controlled sequentially by the automated system with a plasma load corresponding to 21 g of IgG per L of gel, a contact time of 3 to 4 minutes, at an unmodified plasma adsorption pH of 7.1 to 7.8, the elution being performed with acetic acid solution (pH 3.0). The gel was regenerated after each chromatography.

The working flow rate was 76.4 mL/min, which corresponds to a contact time of 3.3 min during the adsorption. The multicolumn affinity chromatography step sequence is summarized in Table 2 below.

TABLE 2
Multicolumn affinity chromatography steps

| Step | Solution | Volume | Remarks |
|---|---|---|---|
| Equilibration | Equilibration | 2 CV | (7.4 ± 0.2) |
| | buffer | | |
| Adsorption | Plasma (0.2 | 35.3 L and 28.4 L | Collection of the |
| | μm filtered) | according to the test | unadsorbed fraction |
| Wash | Equilibration | 2 CV | Until return to |
| | buffer | | baseline |
| Pre-elution | 10 mM sodium | 2 CV | |
| | citrate, 2M | | |
| | NaCl | | |
| Wash 2 | Equilibration | 2 CV | |
| | buffer | | |
| Elution | Acetic acid | 4 CV | Collection until |
| | (pH 3.0) | | return to baseline. |
| Regeneration | Urea | 2 CV | / |
| Column re- | Equilibration | 2 CV | (7.4 ± 0.2) |
| equilibration | buffer | | |
| Storage | 20% ethanol | At least 2 CV after | at the end of the |
| | | passage of 4 CV of | cycle |
| | | purified water. | |

CV: column volume

The characteristics of the IgG-depleted plasma fraction are:

Human albumin at 16 g/L, pH=6.5, conductivity of 12 mS/cm, in 0.01 M citrate buffer.

The composition of the buffer solutions used during the various steps of the multicolumn ion-exchange chromatography process for purifying albumin is summarized in Table 3 below.

TABLE 3
Buffer solutions for HEA-HyperCel multicolumn
ion-exchange chromatography.

| | | Flow rates |
|---|---|---|
| Phase | Buffer/product | (mL/min) |
| Pre- | 0.1M citrate, 0.1M | 8.3 |
| equilibration | NaCl, adjusted to | |
| | pH = 6.5 | |
| Equilibration | 0.01M citrate, 0.1M | 8.3 |
| | NaCl, adjusted to | |
| | pH = 6.5 | |
| Injection | Starting product | 1.75 |
| Serial wash | 0.01M citrate, 0.1M | 2.1 |
| | NaCl, adjusted to | |
| | pH = 6.5 | |
| Elution | 0.01M citrate and | 8.3 |
| | 0.1M NaCl, pH = 3.9 | |
| Regeneration | 0.01M citrate, 0.1M | 8.3 |
| | NaCl, pH = 3 | |
| Sanitisation | 1M NaOH | 8.3 |

Ion-Exchange Chromatography Gels for Capturing Albumin:

For the multicolumn chromatography of albumin, a HyperCel mixed-mode salt-tolerant gel was used.

Five radial 10-mL columns in series were used in combination with a BioSc Lab automated system (Novasep). Six 164-minute cycles are performed, i.e., about 17 hours of continuous operation.

The multicolumn chromatography conditions applied in this example made it possible to confirm a virtually complete capture of the albumin, very little albumin remaining in the unadsorbed fraction. Table 4 below summarizes the yields in each fraction collected and the minimum purity achieved in the purified fraction of interest.

TABLE 4
Albumin yield and estimation of purity
by SDS-PAGE electrophoresis

| Unadsorbed | Eluate | Regeneration | Purity in |
|---|---|---|---|
| fraction yield | yield | yield | the eluate |
| 3.4% | 87.7% | 8.9% | 80% |

Only about 3.4% of the albumin was not adsorbed. An electrophoretic purity of about 80% is observed. After elution at pH=3.9, an 88% yield was obtained. The HEA eluate is then neutralized at pH 6.5 by adding 0.5 N NaOH solution. A sample is taken (referred to as “HEA-HyperCel adjusted pH eluate 1”).

The unretained fraction can then be passed through a column dedicated to the purification of fibrinogen.

Mixed-Mode and Ion-Exchange Chromatography Gels for Capturing Albumin Contaminants:

In order to increase the albumin purity of the preparation, it is possible to specifically adsorb the proteins co-purified during HEA chromatography.

A conventional ion-exchange gel (Macro-Prep DEAE), in which the absence of reactivity to albumin at pH 6.5 was observed beforehand, was used first. Passage of the “HEA-HyperCel adjusted pH eluate 1” fraction through this gel was collected in its entirety with a slight dilution due to volume displacement by the buffer (0.01 M citrate, 0.1 M NaCl, adjusted to pH=6.5). A sample of the unadsorbed fraction (referred to as “Macro-Prep DEAE NA+L”) was taken. The albumin was re-concentrated at 40 g/L on an ultrafiltration membrane (Biomax 10 kD, Merck Millipore) before passage on a second chromatography gel capturing albumin contaminants also at pH 6.5. The MEP HyperCel gel was used because albumin does not interact with the mercapto-ethyl-pyridine group very slightly ionic at this pH. Adsorptions by hydrophobic interactions are investigated on the pyridine ring in particular that targeted against residual immunoglobulins and/or other proteins such as transferrin, ferritin and haptoglobin. After passage of the concentrated solution on the gel, a slight dilution due to volume displacement by the buffer (0.01 M citrate, 0.1 M NaCl, adjusted to pH=6.5) was performed. A sample of the unadsorbed fraction was taken (referred to as “MEP HyperCel NA+L”). Finally, the solution was stabilized by adding sodium caprylate (3 g/L) and was heated at least 2 hours at 60° C. to flocculate the traces of thermosensitive proteins. A last sample was taken after filtration at the 0.2 μm cut-off (referred to as “Heat-treated final”).

TABLE 5
Outputs obtained at the time of the steps of capture
of the contaminants of plasmatic albumin

| | Macro-Prep | | MEP | Heat |
|---|---|---|---|---|
| Step | DEAE | Ultrafiltration | HyperCel | treatment |
| Yield | 100% | 100% | 86.4% | 100% |

By applying such a process, the final albumin yield is 76%. The SDS-PAGE profile corresponding to these tests shows that the pasteurized product has a purity higher than 90% by SDS-PAGE (FIG. 5).

### Example 2

### Characterization of the Albumin Concentrate Obtained in Example 1

The cleavage oxidation state of the albumin was analysed at various steps of the process for continuous capture and purification of albumin from a blood plasma sample:
HEA adjusted pH eluate 1 (HyperCel)
DEAE NA+L (Macro-Prep)
MEP NA (HyperCel)
Heat-treated final

The albumin is injected onto a C4 reverse-phase column and analysed by ESI-MS. Electrospray mass spectrometry gives a mass measurement sufficiently precise to evaluate the heterogeneity of the albumin. The experimental masses are compared with the theoretical masses.

Materials and Methods

The albumin concentrate obtained at the end of the process according to Example 1 (20 μg) is injected onto a C4 reverse-phase column (2.1×150 mm, 300 Å, 1.7 μm) thermostatically-controlled at 60° C.

The mobile phases used are: H₂O with 0.1% TFA (A) and acetonitrile containing 0.1% TFA (B). Elution is carried out by using a gradient of phase B at a flow rate of 200 μL/min.

The eluate is analysed on-line by mass spectrometry on an ESI-Q-TOF apparatus (Synapt, Waters).

Results

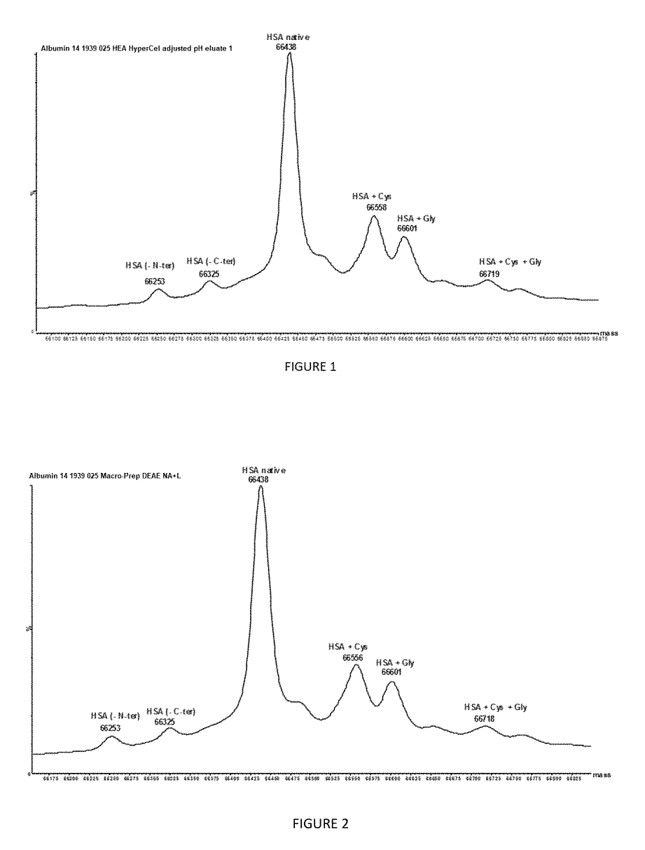

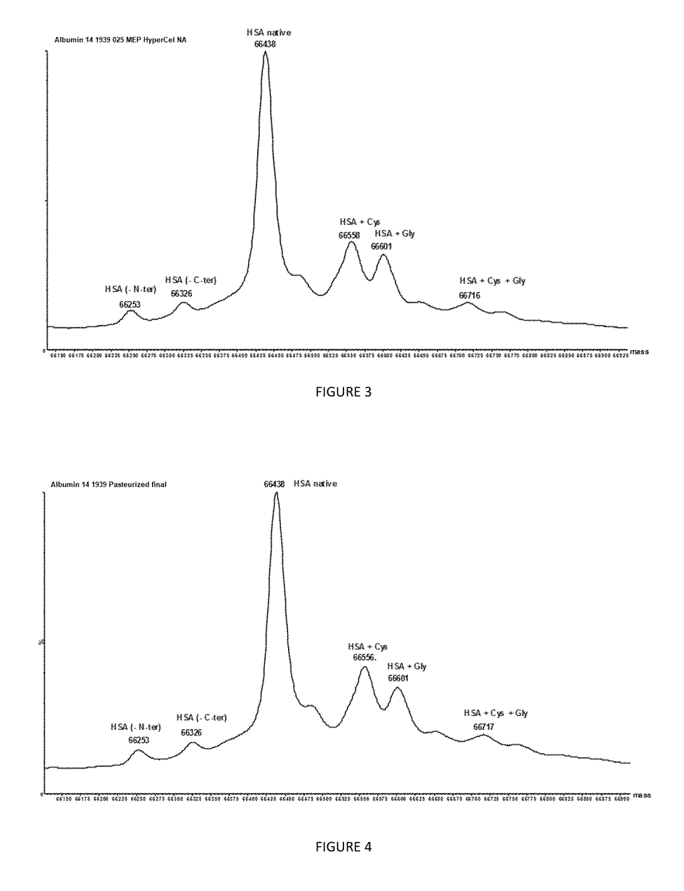

The major experimental mass of 66,438 Da observed for the various steps of the albumin purification process (FIGS. 1-4) corresponds to the mass of native albumin (theoretical mass: 66,438 Da).

The other forms observed on the deconvoluted spectra are minor, the two forms having the largest experimental masses observed of 66,558 Da and 66,601 Da correspond to cysteinylation-modified albumin (theoretical mass: 66,559 Da) and to glycation of albumin (theoretical mass: 66,600 Da), respectively.

The other forms, having experimental masses of 66,253 Da, 66,325 Da and 66,719 Da, correspond to loss of the N-terminal (−Asp₁+Ala₂), to loss of the C-terminal (−Leu₅₈₅), and to cysteinylation+glycation of the albumin, respectively.

Table 6 below summarizes the experimental masses obtained for the deconvoluted mass spectra of the albumin at each purification step.

TABLE 6
Molecular weight of the albumin at the various
steps of the purification process

| | HEA | Macro- | | | |
|---|---|---|---|---|---|
| | HyperCel | Prep | MEP | Heat- | Theo- |
| | adjusted pH | DEAE | HyperCel | treated | retical |
| | eluate 1 | NA + L | NA | final | masses |
| Native human | 66438 | 66438 | 66438 | 66438 | 66438 |
| albumin | | | | | |
| −DA (−N ter) * | 66253 | 66253 | 66253 | 66253 | 66252 |
| −L (−C ter) ** | 66325 | 66325 | 66326 | 66326 | 66325 |
| +Cysteine *** | 66558 | 66556 | 66558 | 66556 | 66559 |
| +Glycation *** | 66601 | 66601 | 66601 | 66601 | 66600 |
| +Cysteine + | 66719 | 66718 | 66716 | 66717 | 66721 |
| glycation *** | | | | | |

* Albumin having a truncated N-terminal end not comprising the N-terminal aspartic acid and alanine residues
** Albumin having a truncated C-terminal end not comprising the C-terminal leucine residue
*** Albumin having a cysteine group and/or an additional glycation

HPLC-MS analysis of the albumin shows that the albumin obtained by the purification process employing a succession of chromatographies and no ethanol precipitation step remains comparable at the various steps of said process.

As summarized in Table 7 below, the major form corresponds to an albumin having a reduced free cysteine (native, unoxidized and untruncated form), the minor forms correspond to the truncated (N-ter and C-ter), oxidized (cysteinylated and cysteinylated/glycated), and glycated forms.

TABLE 7
Percentage of the various forms of albumin in the albumin
composition obtained at the end of the purification process

| | % of the various forms of albumin |
|---|---|
| Native human albumin | 66.4% |
| Truncated (−N-ter and C-ter) | 6.9% (3.5% and 3.4%) |
| Oxidized (+Cysteine | 18.1% (14.1% and 4.0%) |
| and +Cysteine/glycation) | |
| Glycated | 8.6% |

### Example 3

### Characterization of Contaminant Proteins in the Albumin Concentrate

The albumin concentrate of Example 1 was analysed to determine the other plasma proteins present.

Principle

After denaturation, reduction, alkylation and trypsin digestion of the heat-treated final albumin, the peptide mixture obtained is separated and analysed by nanoLC-MS/MS. The result is then reprocessed by the PEAKS Studio software and sent to the databases in order to analyse the accompanying proteins present at the end of the process.

Materials and Methods

The tryptic mixture (200 ng) is injected onto a C18 reverse-phase nanoLC column thermostatically-controlled at 25° C. The mobile phases used are: H₂O with 0.1% TFA (A) and acetonitrile containing 0.1% TFA (B). Elution is carried out by using a gradient of phase

B at a flow rate of 300 nL/min.

The eluate is analysed on-line by mass spectrometry on an LTQ Orbitrap apparatus (Thermo).

Results
Software: PEAKS Studio 7.0
Error tolerance: parent ions 5 ppm, fragment ions 0.1 Da
False-discovery rate: 0.1%
Number of peptides necessary to identify a protein: ≥2 peptides/protein
Fixed PTMs: carbamidomethylation

Variable PTMs: Deamination, Oxidation
Database: HUMAN (04/2015)

Table 8 below presents all the results obtained after processing the result.

TABLE 8
List of proteins present in the heat-
treated final albumin concentrate

| | Cover- | #Pep- | Molecular | |
|---|---|---|---|---|
| Accession | age (%) | tides | weight (Da) | Protein |
| P02768 | 83 | 74 | 69367 | Serum albumin |
| P02787 | 37 | 24 | 77064 | Serotransferrin |
| P01876 | 24 | 6 | 37655 | Ig alpha-1 chain C region |
| P01009 | 10 | 4 | 46737 | Alpha-1-antitrypsin |
| P01834 | 69 | 5 | 11609 | Ig kappa chain C region |
| P01008 | 11 | 5 | 52602 | Antithrombin-III |
| P00738 | 28 | 8 | 45205 | Haptoglobin |
| P01877 | 15 | 4 | 36526 | Ig alpha-2 chain C region |
| P01023 | 3 | 4 | 163290 | Alpha-2-macroglobulin |
| P04217 | 12 | 4 | 54254 | Alpha-1B-glycoprotein |
| P02763 | 9 | 3 | 23512 | Alpha-1-acid glycoprotein 1 |
| P02675 | 8 | 3 | 55928 | Fibrinogen beta chain |
| P02671 | 2 | 2 | 94973 | Fibrinogen alpha chain |
| A0M8Q6 | 32 | 2 | 11303 | Ig lambda-7 chain C |

The albumin concentrate according to the invention has a low level of contaminant proteins, making it advantageous for therapeutic use.

### Example 4

### Preparation of Human Albumin Compositions for Therapeutic Use

Starting with the albumin composition obtained in Example 1, ultrafiltration-concentrated forms (40, 50, 200 and 250 g/L) are prepared, stabilized by sodium caprylate salts, and pasteurized according to methods conventionally used in commercial preparations without limitation of dose or of concentration.

### Example 5

### Comparison of the Albumin Composition Obtained in Example 1 with an Albumin Composition of the Prior Art

The albumin composition obtained in Example 1 and a product of the prior art (Albuminar® from CSL) were analysed by mass spectrometry.

Materials and Methods

The albumin concentrate obtained at the end of the process according to Example 1 (20 μg) was injected onto a C4 reverse-phase column (2.1×150 mm, 300 Å, 1.7 μm) thermostatically-controlled at 60° C.

The mobile phases used are: H₂O with 0.1% TFA (A) and acetonitrile containing 0.1% TFA (B). The elution was performed using a gradient of phase B at a flow rate of 200 μL/min.

The eluate was analysed on-line by mass spectrometry on an ESI-Q-TOF apparatus (Synapt, Waters).

Results

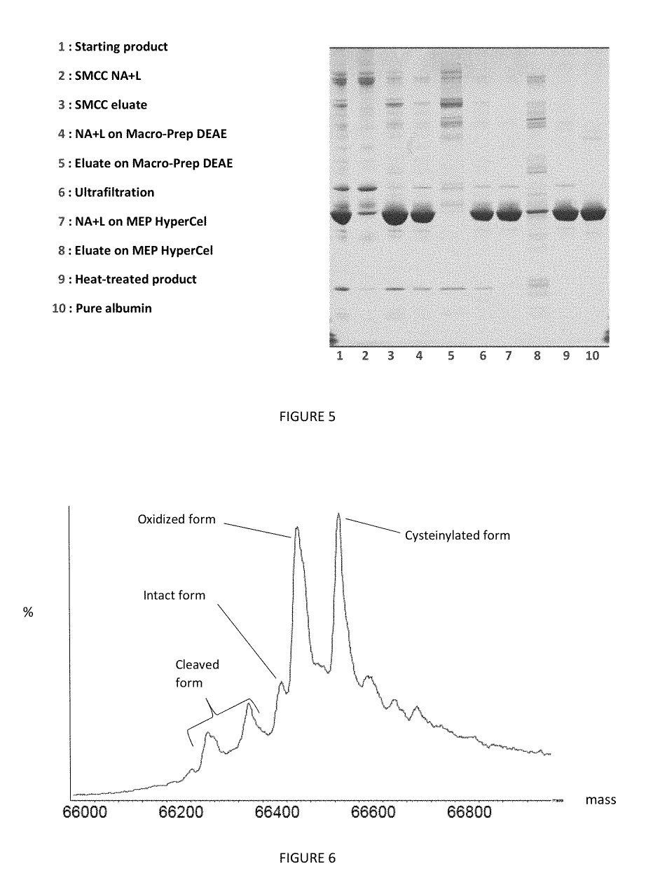

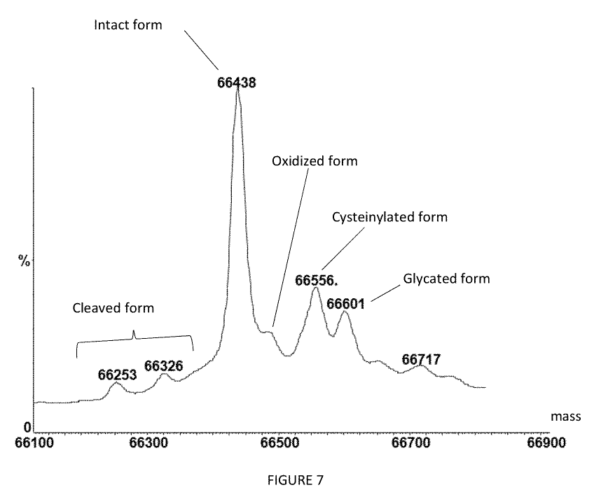

The results obtained are shown in FIG. 6 (spectrum of the albumin composition Albuminar® from CSL Behring) and FIG. 7 (spectrum of the albumin composition according to the invention, obtained in Example 1).

Analysis of the spectra shows a high proportion of oxidized and cysteinylated forms in the product Albuminar®, compared with the composition according to the invention.

These results confirm that the composition according to the invention comprises a larger amount of unoxidized, uncysteinylated and N/C-ter uncleaved albumin than the composition of the prior art.

1-15. (canceled)

16. A liquid composition of human albumin for therapeutic use, wherein said albumin has been obtained by a purification process of blood plasma deprived of precipitation step and wherein at least 50% of the albumin has a molecular weight of 66,438 Da, plus or minus 5 Da, said composition further comprising a sodium salt.

17. The liquid composition of human albumin for therapeutic use according to claim 16, comprising between 10 and 300 mg/mL albumin.

18. The liquid composition of human albumin for therapeutic use according to claim 16, comprising between 40 and 50 mg/mL albumin.

19. The liquid composition of human albumin for therapeutic use according to claim 16, comprising between 200 and 250 mg/mL albumin.

20. The liquid composition of human albumin for therapeutic use according to claim 16, comprising less than 30% albumin in oxidized form.

21. The liquid composition of human albumin for therapeutic use according to claim 16, comprising less than 20% albumin selected from albumin having a truncated C-terminal end and albumin having N-terminal end.

22. The liquid composition of human albumin for therapeutic use according to claim 16, said composition comprising between 2.5 and 3.5 mg/mL sodium salt.

23. The liquid composition of human albumin for therapeutic use according to claim 16, wherein the albumin has been obtained by a purification process deprived of cryoprecipitation step.

24. The liquid composition of human albumin for therapeutic use according to claim 16, wherein the albumin has been obtained by a purification process comprising positive chromatography followed by negative chromatography.

25. The liquid composition of human albumin for therapeutic use according to claim 24, said composition further comprising at least one plasma protein among transferrin, immunoglobulin G, haptoglobin.

26. A lyophilized composition obtained by lyophilization of a liquid composition of human albumin according to claim 16.

27. Treatment of a disorder selected from renal impairment, hepatic impairment, neurodegenerative disease, hypovolemic shock, adult respiratory distress syndrome (ARDS), haemolytic disease of the new-born, wherein the composition according to claim 16 is administered to a patient in need thereof.

28. A process for preparing a liquid composition of human albumin for therapeutic use according to claim 16, comprising purification steps wherein blood plasma is subjected to a positive chromatography followed by a negative chromatography.

29. A process for preparing a liquid composition of human albumin for therapeutic use according to claim 16, comprising the fractionation steps of blood plasma consisting in:
a) Subjecting blood plasma to anion-exchange chromatography at pH 5 or higher;
b) Subjecting the eluted fraction derived from step a) to a first ion-exchange or mixed-mode chromatography,
c) Subjecting the unretained fraction derived from step b) to a second ion-exchange or mixed-mode chromatography.

30. The process of claim 29 further comprising the step consisting in:
d) Subjecting the unretained fraction derived from step c) to heat treatment, at a temperature of 50° C. to 70° C., for a period of 1 hour to 15 hours.

31. The liquid composition of human albumin for therapeutic use according to claim 17, said composition comprising between 2.5 and 3.5 mg/mL sodium salt.

32. The liquid composition of human albumin for therapeutic use according to claim 18, said composition comprising between 2.5 and 3.5 mg/mL sodium salt.

33. The liquid composition of human albumin for therapeutic use according to claim 19, said composition comprising between 2.5 and 3.5 mg/mL sodium salt.

34. The liquid composition of human albumin for therapeutic use according to claim 20, said composition comprising between 2.5 and 3.5 mg/mL sodium salt.

35. The liquid composition of human albumin for therapeutic use according to claim 21, said composition comprising between 2.5 and 3.5 mg/mL sodium salt.
